(19) United States
(12) Patent Application Publication
(10) Pub. No.: US 2026/0158293 A1
(43) Pub. Date: Jun. 11, 2026

(54) METHOD AND APPARATUS TO FACILITATE PATIENT COACHING

(71) Applicant: Siemens Healthineers International AG, Steinhausen (CH)

(72) Inventors: Erwan Guerin, Saint-Louis (FR); Viola Sugaya, Wettingen (CH); Julien Gueuning, Brugg (CH)

(21) Appl. No.: 18/977,248

(22) Filed: Dec. 11, 2024

(51) Int. Cl. A61N 5/10

(52) U.S. Cl. CPC A61N 5/1037; A61N 5/1048; A61N 2005/1092

(57) ABSTRACT

A control circuit can present to a breathing patient a first illumination that corresponds to a first light modulation to indicate when normal breathing is allowed during an energy-exposure treatment session, present to the breathing patient a second illumination that corresponds to a second light modulation that is different from the first light modulation to indicate that controlled breathing is presently required during the energy-exposure treatment session, and present to the breathing patient a third illumination that corresponds to a third light modulation that is different from both the first light modulation and the second light modulation to indicate that normal breathing will soon conclude and that controlled breathing will soon begin.

### TECHNICAL FIELD

These teachings relate generally to treating a patient's planning target volume with energy pursuant to an energy-based treatment plan and more particularly to administering an energy-based treatment plan.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called radiation treatment plan often serves in the foregoing regards.

An energy-exposure treatment plan, such as a radiation treatment plan, typically comprises specified values for each of a variety of treatment-platform parameters during each of a plurality of sequential fields. Treatment plans for radiation treatment sessions are often automatically generated through a so-called optimization process. As used herein, “optimization” will be understood to refer to improving a candidate treatment plan without necessarily ensuring that the optimized result is, in fact, the singular best solution. Such optimization often includes automatically adjusting one or more physical treatment parameters (often while observing one or more corresponding limits in these regards) and mathematically calculating a likely corresponding treatment result (such as a level of dosing) to identify a given set of treatment parameters that represent a good compromise between the desired therapeutic result and avoidance of undesired collateral effects.

In most cases a patient will be awake while therapeutic radiation is administered. In some cases, the patient will be asked to exhibit a particular behavior beyond simply remaining still while radiation is administered. As one example, the patient may be asked to halt their breathing and hold their breath from time to time in order to help ensure that the radiation target is properly located per the requirements of the radiation treatment plan.

### BRIEF DESCRIPTION OF DRAWINGS

The above needs are at least partially met through provision of the method and apparatus to facilitate patient coaching described in the following detailed description, particularly when studied in conjunction with the drawings, wherein:

FIG. 1 comprises a block diagram as configured in accordance with various embodiments of these teachings;

FIG. 2 comprises a perspective view as configured in accordance with various embodiments of these teachings;

FIG. 3 comprises a side elevational view as configured in accordance with various embodiments of these teachings;

FIG. 4 comprises a front plan view as configured in accordance with various embodiments of these teachings;

FIG. 5 comprises a block diagram as configured in accordance with various embodiments of these teachings;

FIG. 6 comprises a flow chart as configured in accordance with various embodiments of these teachings; and

FIG. 7 comprises a timing diagram as configured in accordance with various embodiments of the invention.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, these various embodiments can be carried out during an energy-exposure treatment session of a target volume in a breathing patient, wherein the target volume moves in response to that breathing. A control circuit can be configured to present to the breathing patient a first illumination that corresponds to a first light modulation to indicate when normal breathing is allowed during the energy-exposure treatment session. The control circuit can be further configured to present to the breathing patient a second illumination that corresponds to a second light modulation (that is different from the first light modulation) to indicate that controlled breathing (such as holding one's breath) is required during the energy-exposure treatment session. And the control circuit can be yet further configured to present to the breathing patient a third illumination that corresponds to a third light modulation (that is different from both the first light modulation and the second light modulation) to indicate that normal breathing will soon conclude and that controlled breathing will soon begin (to thereby provide, for example, an opportunity for the patient to inhale prior to then needing to hold their breath).

By one approach, the aforementioned first, second, and third illumination can each comprise ambient illumination. As used herein, ambient illumination will be understood to comprise a type of lighting that provides a dispersed (often more uniform) level of illumination throughout a given space and where the source of the lighting is physically obscured from view. Ambient lighting accordingly does not include, for example, directed light such as spotlighting that constitutes a strong, focused beam of light on a specific area.

The aforementioned light modulation can comprise one or more of, for example, a color-choice modulation, a light-intensity modulation, and a flashing-light modulation. As an illustrative example, and without intending to suggest any limitations with respect to these teachings, the aforementioned first light modulation can comprise use of a green color, the second light modulation can comprise use of a red color, and the third light modulation can comprise use of at least one of a yellow color and an orange color.

By one approach, one or more of the aforementioned illuminations can be accompanied by acoustic content. By one approach, different unique acoustic content can be provided for each of the three different aforementioned light modulations.

By one approach, the aforementioned control circuit can access image information of the breathing patient and can be configured to determine when to present to the breathing patient any of the aforementioned illuminations as a function, at least in part, of the accessed image information. So configured, the coaching information provided by these teachings can be rendered in an automatic manner that relieves the attending clinicians from attending to or otherwise monitoring that task.

By one approach, these teachings will accommodate providing and using a lighting system that is capable of providing the aforementioned first, second, and third illuminations in the treatment setting in accordance with their corresponding light modulations. By one approach, these teachings will accommodate a ceiling-mounted frame that supports the lighting system. In a case where the illumination comprises ambient illumination, the ceiling-mounted frame can be configured to dispose the lighting system at least 1.5 meters distant from (and above) the breathing patient.

These teachings will accommodate a wide variety of form factors for a ceiling-mounted frame. By one useful approach, the ceiling-mounted frame can comprise an arc-shaped frame.

By one approach, one or more image-capture apparatuses can also be supported by the ceiling-mounted frame. In this case, the image-capture apparatuses can be aimed and configured to capture images of the breathing patient and/or the positions and movements of various elements of the radiation treatment platform such as gantry positions that are then utilized as described herein.

By one approach, in lieu of the foregoing or in combination therewith, these teachings will also accommodate operably coupling at least one audio transducer to the ceiling-mounted frame. That at least one audio transducer can then be utilized to provide the acoustic content described herein.

By one approach, these teachings can comprise a computer program that itself comprises instructions that, when the computer program is executed by a computer, causes the computer to carry out one or more of the various actions, steps, and/or functions set forth herein. As one example in these regards, these teachings will accommodate a non-transitory computer-readable medium comprising instructions stored thereon for use during an energy-exposure treatment session of a target volume in a breathing patient, which target volume moves in response to the breathing, which instructions, when executed on a processor, perform the steps of presenting to the breathing patient a first illumination that corresponds to a first light modulation to indicate when normal breathing is allowed during the energy-exposure treatment session, presenting to the breathing patient a second illumination that corresponds to a second light modulation that is different from the first light modulation to indicate that controlled breathing is required during the energy-exposure treatment session, and presenting to the breathing patient a third illumination that corresponds to a third light modulation that is different from both the first light modulation and the second light modulation to indicate that normal breathing will soon conclude and that controlled breathing will soon begin.

So configured, these teachings can facilitate a mechanism for successfully and efficaciously coaching a patient during an energy-exposure treatment session, and in particular as regards directing the patient's breathing. This coaching can be highly automated if desired, and can thereby free the clinicians from both setup requirements and during-treatment responsibilities that might otherwise divert their attention from other tasks. Accordingly, these teachings can help to shorten the overall amount of time required to conduct a radiation treatment for an energy-exposure target whose position will change as a patient breathes. That result, in turn, can help increase throughput for the energy-exposure treatment platform and thereby allow a greater number of patients to be treated within a given period of time.

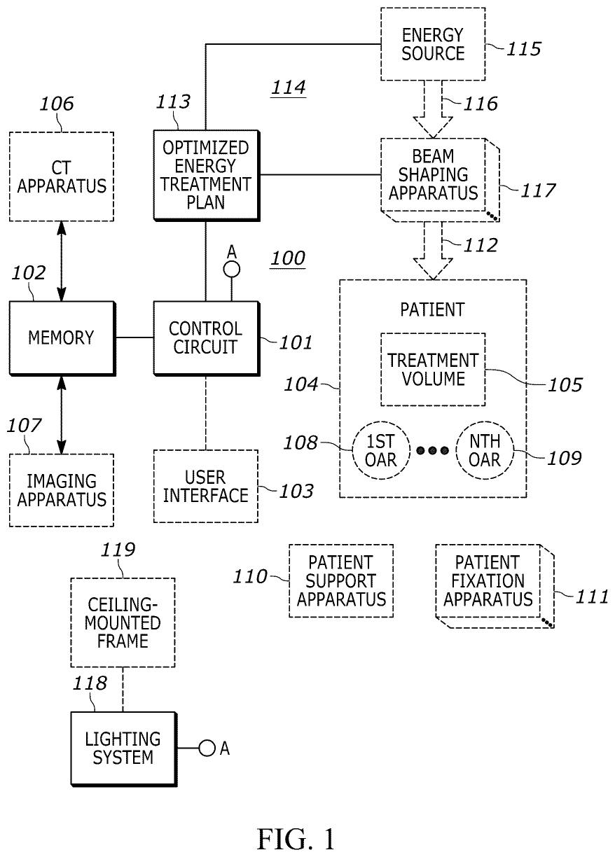

These and other benefits may become clearer upon making a thorough review and study of the following detailed description. Referring now to the drawings, and in particular to FIG. 1, an illustrative apparatus 100 that is compatible with many of these teachings will first be presented.

In this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

It will be appreciated that the control circuit 101 may comprise a single integrated platform or may comprise a plurality of such circuits that work in cooperation with one another.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101). As with the control circuit 101, the memory 102 may comprise a singular structure or may comprise a plurality of memory platforms that collectively comprise the “memory” of this apparatus 100.

In addition to information such as optimization information for a particular patient and information regarding a particular energy-exposure treatment platform as described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

By one optional approach the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless platforms, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized energy-exposure treatment plan (such as, for example, an optimized radiation treatment plan 113). This energy-exposure treatment plan typically comprises specified values for each of a variety of treatment-platform parameters during each of a plurality of sequential exposure fields. In this case the energy-based treatment plan is generated through an optimization process, examples of which are provided further herein.

By one approach the control circuit 101 can operably couple to an energy-based treatment platform 114 that is configured to deliver therapeutic energy 112 to a corresponding patient 104 having at least one treatment volume 105 and also one or more organs-at-risk (represented in FIG. 1 by a first through an Nth organ-at-risk 108 and 109) in accordance with the optimized energy-based treatment plan 113. These teachings are generally applicable for use with any of a wide variety of energy-based treatment platforms/apparatuses. In a typical application setting the energy-based treatment platform 114 will include an energy source such as a radiation source 115 of ionizing radiation 116.

By one approach this radiation source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the radiation source 115 along that arcuate pathway, and may accordingly control when the radiation source 115 starts moving, stops moving, accelerates, de-accelerates, and/or a velocity at which the radiation source 115 travels along the arcuate pathway.

As one illustrative example, the radiation source 115 can comprise, for example, a radio-frequency (RF) linear particle accelerator-based (linac-based) x-ray source. A linac is a type of particle accelerator that greatly increases the kinetic energy of charged subatomic particles or ions by subjecting the charged particles to a series of oscillating electric potentials along a linear beamline, which can be used to generate ionizing radiation (e.g., X-rays) 116 and high energy electrons.

A typical energy-based treatment platform 114 may also include one or more support apparatuses 110 (such as a couch) to support the patient 104 during the treatment session, one or more patient fixation apparatuses 111, a gantry or other movable mechanism to permit selective movement of the radiation source 115, and one or more energy-shaping apparatuses (for example, beam-shaping apparatuses 117 such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

In this illustrative example, the radiation treatment platform 114 also includes a lighting system 118. This lighting system 118 is configured to provide at least a first illumination in the treatment setting that corresponds to a first light modulation, a second illumination in the treatment setting that corresponds to a second light modulation, and a third illumination in the treatment setting that corresponds to a third light modulation, wherein the first, second, and third light illuminations are all different from one another.

The lighting system 118 can operably couple to the aforementioned control circuit 101. So configured, the control circuit 101 can use the lighting system 118 to present to a breathing patient 104 the various available illuminations during an energy-exposure treatment session per the teachings provided herein.

By one approach, the aforementioned first, second, and third illuminations each comprise ambient illumination as versus direct illumination. The illumination sources themselves can comprise monochromatic light sources and/or selectively-polychromatic light sources as desired.

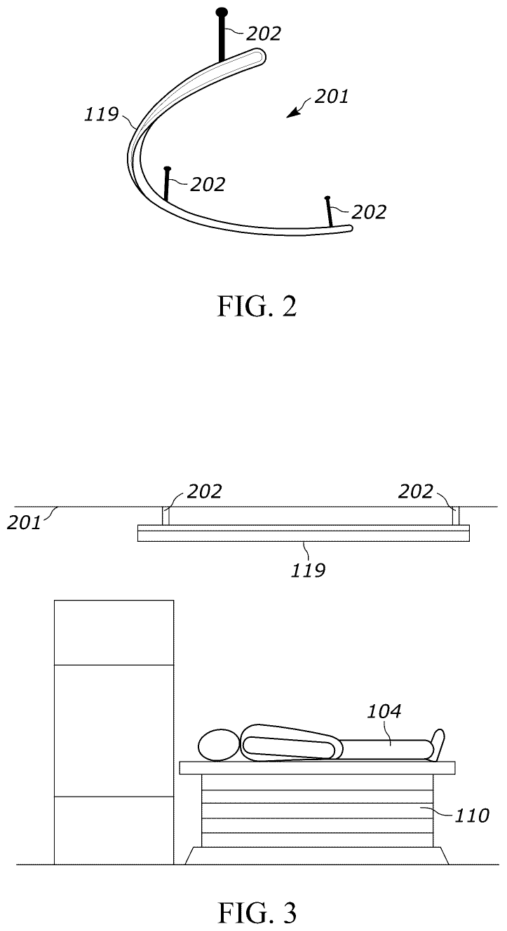

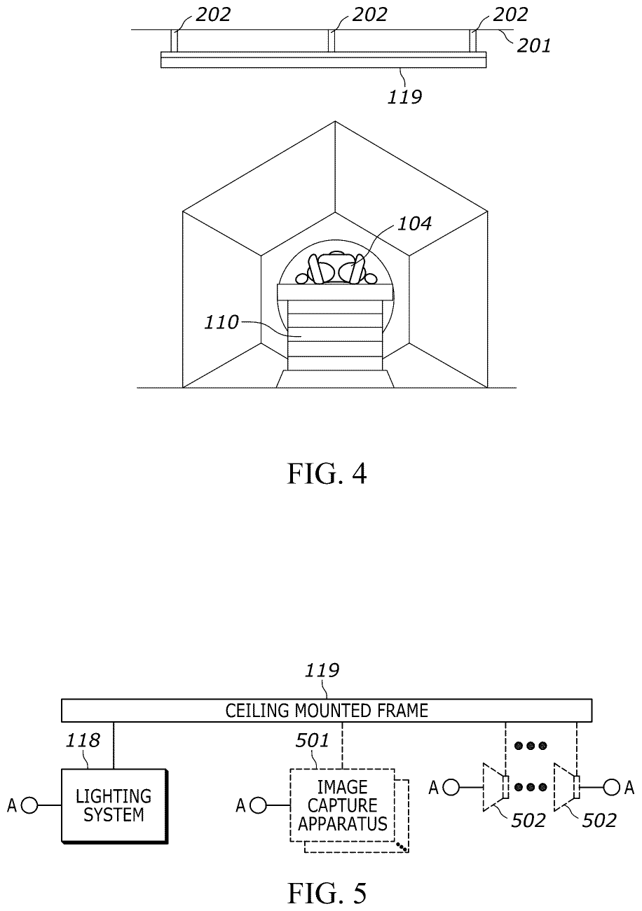

By one optional approach, part or all of the lighting system 118 can be connected to and supported by a ceiling-mounted frame 119. FIGS. 2 through 4 provide an illustrative example in these regards. In this example, the ceiling-mounted frame 119 is arc-shaped and connects to a ceiling 201 (such as the ceiling in the room that contains the patient support apparatus 110) by way of three struts 202 that dispose the ceiling-mounted frame 119 a short distance (such as 1 to 10 cm) away from the ceiling 201. In this illustrative example, the top of the arc is positioned more towards the patient's head then towards the patient's feet.

The light sources for the lighting system 118 can be positioned, for example, on the upper side and/or at least partially on the side edges of the ceiling-mounted frame 119 such that the light emitted by the lighting system 118 is directed, at least in large part (such as at least 60%, 80%, or 90%) towards the ceiling 201. So configured, the ceiling-mounted frame 119 can dispose the lighting system 118 at least 1.5 meters above and distant from the breathing patient 104.

Referring to FIG. 5, by one optional approach, one or more image-capture apparatuses 501 can also be supported by the ceiling-mounted frame 119. These image-capture apparatuses 501 may comprise, for example, digital cameras that capture still images or video content. These one or more image-capture apparatuses 501 can include at least two image-capture apparatuses that are aimed and configured to capture images of the breathing patient 104, preferably at an angle sufficient to observe movement of the patient that results from and that corresponds to the patient's breathing. The positioning and orientation of the field of view and/or any zooming capability can be controlled by the control circuit 101 if desired. Image information provided by these image capture apparatuses 501 can be utilized by the control circuit 101 as described herein.

With continued reference to FIG. 5, these teachings will also accommodate supporting at least one audio transducer 502 by the ceiling-mounted frame 119. These one or more audio transducers 502 can be operably coupled to the control circuit 101. The control circuit 101 can be configured to provide acoustic content, or to otherwise control the delivery of acoustic content, to the breathing patient 104 via the at least one audio transducer 502 in accompaniment to presenting one or more of the various illuminations described herein.

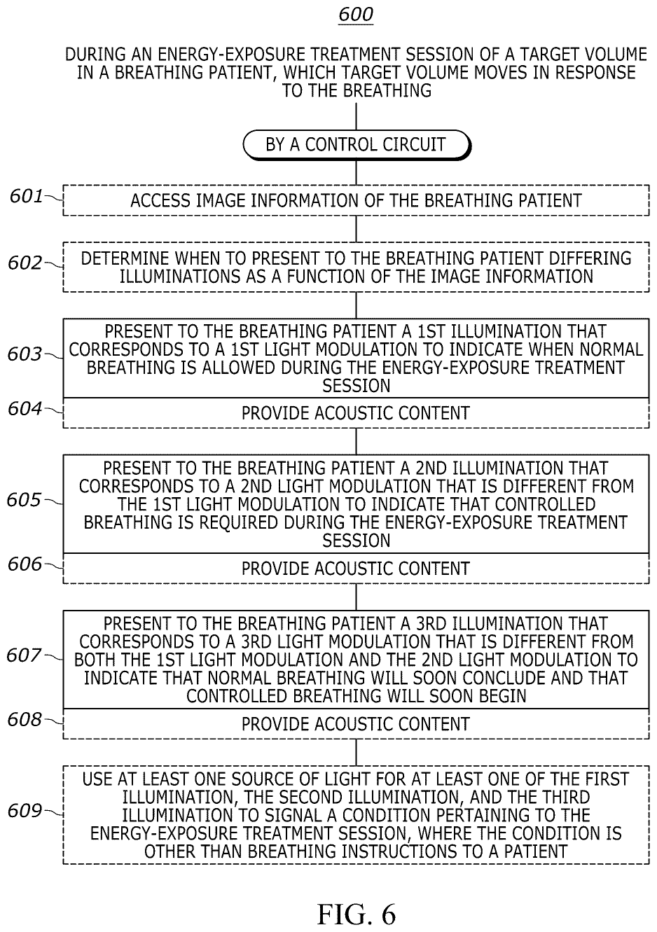

Referring now to FIG. 6, a process 600 that can be carried out, for example, in conjunction with the above-described application setting (and more particularly via the aforementioned control circuit 101) will be described. Generally speaking, this process 600 serves to provide coaching information to a patient 104 during an energy-exposure treatment session to thereby help the patient 104 appropriately control their breathing in a way that accommodates the energy-exposure treatment plan.

At block 601, by one optional approach this process 600 can provide for accessing imaging information of the breathing patient 104. This image information can be sourced, for example, by the above-described image capture apparatus(es) 501. At optional block 602, the control circuit 101 can then determine when to present to the breathing patient 104 one or more of the illuminations described herein as a function of that image information. As one example in these regards, the image information may comprise images that are captured over time (such as video content or snapshots taken at short periodic intervals (such as every 0.1 seconds, 0.2 seconds, 0.3 seconds, or at any desired rate of periodicity)), which images can be compared to one another to determine, for example, when the patient has fully exhaled or inhaled, when they are breathing normally, when they are holding their breath, and so forth. Such information can then be used to trigger operation of the illumination options. So configured, the present teachings can be carried out with little or no real-time supervision by attending clinicians.

At block 603, this process 600 can present to the breathing patient 104 a first illumination that corresponds to a first light modulation to indicate when normal breathing is allowed during the energy-exposure treatment session. The light modulation can comprise, for example, one or more of a color-choice modulation, a light-intensity modulation, and/or a flashing-light modulation. As an illustrative example, this first light modulation can comprise employing a green color for the light.

If desired, and referring to optional block 604, this process 600 will accommodate also providing acoustic content to the breathing patient 104 in accompaniment to providing the aforementioned first illumination. This acoustic content can be unique to be being used only in accompaniment to providing the first illumination. These teachings will accommodate any of a wide variety of acoustic sounds, including single-note tones, short monophonic or polyphonic melodies, spoken words, and so forth.

At block 605, the control circuit 101 presents to the breathing patient 104 a second illumination that corresponds to a second light modulation (such as a red colored illumination) that is different from the first light modulation to indicate that controlled breathing (such as holding one's breath) is required at this time during the energy-exposure treatment session. This second illumination can be provided throughout the duration of the time that such controlled breathing is required.

As before, and as indicated by optional block 606, acoustic content that is unique for use with the second illumination can also be provided in accompaniment to the second illumination.

At block 607, the control circuit 101 presents to the breathing patient 104 a third illumination that corresponds to a third light modulation (such as use of at least one of a yellow color or an orange color) that is different from both the first light modulation and the second light modulation to indicate that normal breathing will soon conclude and that controlled breathing will soon begin. This third illumination may be presented, for example, for fixed durations of time such as one second, two seconds, or such other duration as may be appropriate to the application setting.

And again, and as indicated by optional block 608, acoustic content that is unique for use with the third illumination can be provided in accompaniment to the third illumination.

So configured, a patient 104 can be intuitively and successfully coached as regards their breathing behavior during an energy-exposure treatment session. The benefits of these teachings are achieved without requiring placement of any visual or acoustic interfaces in close proximity to the patient (such as, for example, within 0.5 meters or less), thereby avoiding physical set-up requirements for the attending clinicians and also obviating potential collision opportunities with other moving elements of the treatment facility platform 114.

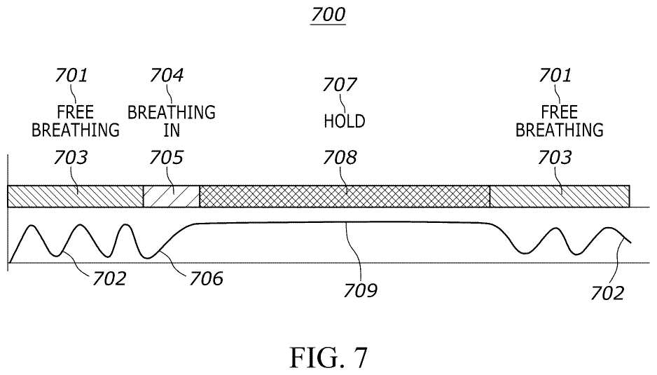

FIG. 7 provides an illustrative example 700 in these regards. During a period of time when free breathing 701 is permitted, the patient 104 can breathe normally 702 and the aforementioned first illumination 703 can be presented to the patient 104. When the session is about to transition 704 to a controlled breathing mode, the aforementioned third illumination 705 can be presented to the patient 104 to prompt the patient 104 to breathe in 706 in preparation for holding their breath. During the subsequent controlled breathing mode 707, the aforementioned second illumination 708 is presented to the patient 104 to remind and prompt the patient 104 to hold and to continue holding their breath 709. When the controlled breathing mode 707 concludes, the process returns to the free breathing mode 701 as signaled by presenting the 1st illumination 703 to the patient 104. The aforementioned cycle can be repeated as often as necessary to accommodate the needs of the particular energy-exposure treatment session.

Referring again to FIG. 6, by one optional approach, and as shown at block 609, these teachings will accommodate using the lighting system 118 to signal a condition pertaining to the energy-exposure treatment session other than signaling breathing instructions to the patient. For example, a flashing light may provide information to an attending clinician that the treatment session is about to conclude, or to indicate some other condition of potential concern (such as a potential or actual collision). As the illumination provided by the lighting system 118 is ambient, the information so conveyed to the clinician is achieved without requiring the clinician to occasionally or constantly monitor a user interface.

Further aspects of these teachings are provided by the subject matter of the following clauses (where it will be understood that any of these clauses can be combined with any one of more of the other clauses as appropriate).

Clause 1. A method for use during an energy-exposure treatment session of a target volume in a breathing patient, which target volume moves in response to the breathing, the method comprising: by a control circuit: presenting to the breathing patient a first illumination that corresponds to a first light modulation to indicate when normal breathing is allowed during the energy-exposure treatment session; presenting to the breathing patient a second illumination that corresponds to a second light modulation that is different from the first light modulation to indicate that controlled breathing is required during the energy-exposure treatment session; presenting to the breathing patient a third illumination that corresponds to a third light modulation that is different from both the first light modulation and the second light modulation to indicate that normal breathing will soon conclude and that controlled breathing will soon begin.

Clause 2. The method of clause 1 wherein the first, second, and third illumination each comprise ambient illumination.

Clause 3. The method of clause 1 wherein the first light modulation comprises at least one of: a color-choice modulation; a light-intensity modulation; a flashing-light modulation.

Clause 4. The method of clause 1 wherein the first light modulation comprises use of a green color and the second light modulation comprises use of a red color.

Clause 5. The method of clause 4 wherein the third light modulation comprises use of at least one of a yellow color and an orange color.

Clause 6. The method of clause 1 further comprising: accessing image information of the breathing patient; determining when to present to the breathing patient at least one of the first illumination and the second illumination as a function of the image information.

Clause 7. The method of clause 1 further comprising: providing acoustic content to the breathing patient in accompaniment to at least one of presenting to the breathing patient the first illumination, the second illumination, and the third illumination.

Clause 8. The method of clause 7 wherein providing acoustic content to the breathing patient in accompaniment to at least one of presenting to the breathing patient the first illumination, the second illumination, and the third illumination comprises providing acoustic content to the breathing patient in accompaniment to all three of presenting to the breathing patient the first illumination, the second illumination, and the third illumination.

Clause 9. The method of clause 8 wherein providing the acoustic content to the breathing patient in accompaniment to all three of presenting to the breathing patient the first illumination, the second illumination, and the third illumination comprises: providing first acoustic content to the breathing patient to accompany presenting to the breathing patient the first illumination; providing second acoustic content to the breathing patient to accompany presenting to the breathing patient the second illumination, which second acoustic content is different from the first acoustic content; providing third acoustic content to the breathing patient to accompany presenting to the breathing patient the third illumination, which third acoustic content is different from both the first acoustic content and the second acoustic content.

Clause 10. The method of clause 1 further comprising using at least one source of light for at least one of the first illumination, the second illumination, and the third illumination to signal a condition pertaining to an energy-exposure treatment session, where the condition is other than breathing instructions to a patient.

Clause 11. An apparatus configured for use in a treatment setting during an energy-exposure treatment session of a target volume in a breathing patient, the apparatus comprising: a lighting system capable of providing a first illumination in the treatment setting that corresponds to a first light modulation, a second illumination in the treatment setting that corresponds to a second light modulation, and a third illumination in the treatment setting that corresponds to a third light modulation, wherein the first, second, and third light modulations are all different from one another; a control circuit operably coupled to the lighting system, the control circuit configured to: use the lighting system to present to the breathing patient the first illumination to indicate to the breathing patient when normal breathing is presently allowed during the energy-exposure treatment session; use the lighting system to present to the breathing patient the second illumination to indicate to the breathing patient that controlled breathing is presently required during the energy-exposure treatment session; use the lighting system to present to the breathing patient the third illumination to indicate that normal breathing will soon conclude and that controlled breathing will soon begin.

Clause 12. The apparatus of clause 11 wherein the first, second, and third illumination each comprise ambient illumination.

Clause 13. The apparatus of clause 11 further comprising: a ceiling-mounted frame that supports the lighting system.

Clause 14. The apparatus of clause 13 wherein the ceiling-mounted frame disposes the lighting system at least 1.5 meters distant from the breathing patient.

Clause 15. The apparatus of clause 13 wherein the ceiling-mounted frame comprises an arc-shaped frame.

Clause 16. The apparatus of clause 13 wherein the apparatus further comprises at least one image-capture apparatus that is also supported by the ceiling-mounted frame.

Clause 17. The apparatus of clause 16 wherein the at least one image-capture apparatus comprises at least two image-capture apparatuses that are aimed and configured to capture images of the breathing patient.

Clause 18. The apparatus of clause 13 wherein the apparatus further comprises at least one audio transducer that is also supported by the ceiling-mounted frame.

Clause 19. The apparatus of clause 18 wherein the at least one audio transducer is operably coupled to the control circuit, and wherein the control circuit is configured to provide acoustic content to the breathing patient via the at least one audio transducer in accompaniment to at least one of presenting to the breathing patient the first illumination, the second illumination, and the third illumination.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

1. A method for use during an energy-exposure treatment session of a target volume in a breathing patient, which target volume moves in response to the breathing, the method comprising:
by a control circuit:
presenting to the breathing patient a first illumination that corresponds to a first light modulation to indicate when normal breathing is allowed during the energy-exposure treatment session;
presenting to the breathing patient a second illumination that corresponds to a second light modulation that is different from the first light modulation to indicate that controlled breathing is required during the energy-exposure treatment session;
presenting to the breathing patient a third illumination that corresponds to a third light modulation that is different from both the first light modulation and the second light modulation to indicate that normal breathing will soon conclude and that controlled breathing will soon begin.

2. The method of claim 1 wherein the first, second, and third illumination each comprise ambient illumination.

3. The method of claim 1 wherein the first light modulation comprises at least one of:
a color-choice modulation;
a light-intensity modulation;
a flashing-light modulation.

4. The method of claim 1 wherein the first light modulation comprises use of a green color and the second light modulation comprises use of a red color.

5. The method of claim 4 wherein the third light modulation comprises use of at least one of a yellow color and an orange color.

6. The method of claim 1 further comprising:
accessing image information of the breathing patient;
determining when to present to the breathing patient at least one of the first illumination and the second illumination as a function of the image information.

7. The method of claim 1 further comprising:
providing acoustic content to the breathing patient in accompaniment to at least one of presenting to the breathing patient the first illumination, the second illumination, and the third illumination.

8. The method of claim 7 wherein providing acoustic content to the breathing patient in accompaniment to at least one of presenting to the breathing patient the first illumination, the second illumination, and the third illumination comprises providing acoustic content to the breathing patient in accompaniment to all three of presenting to the breathing patient the first illumination, the second illumination, and the third illumination.

9. The method of claim 8 wherein providing the acoustic content to the breathing patient in accompaniment to all three of presenting to the breathing patient the first illumination, the second illumination, and the third illumination comprises:
providing first acoustic content to the breathing patient to accompany presenting to the breathing patient the first illumination;
providing second acoustic content to the breathing patient to accompany presenting to the breathing patient the second illumination, which second acoustic content is different from the first acoustic content;
providing third acoustic content to the breathing patient to accompany presenting to the breathing patient the third illumination, which third acoustic content is different from both the first acoustic content and the second acoustic content.

10. The method of claim 1 further comprising using at least one source of light for at least one of the first illumination, the second illumination, and the third illumination to signal a condition pertaining to an energy-exposure treatment session, where the condition is other than breathing instructions to a patient.

11. An apparatus configured for use in a treatment setting during an energy-exposure treatment session of a target volume in a breathing patient, the apparatus comprising:
a lighting system capable of providing a first illumination in the treatment setting that corresponds to a first light modulation, a second illumination in the treatment setting that corresponds to a second light modulation, and a third illumination in the treatment setting that corresponds to a third light modulation, wherein the first, second, and third light modulations are all different from one another;
a control circuit operably coupled to the lighting system, the control circuit configured to:
use the lighting system to present to the breathing patient the first illumination to indicate to the breathing patient when normal breathing is presently allowed during the energy-exposure treatment session;
use the lighting system to present to the breathing patient the second illumination to indicate to the breathing patient that controlled breathing is presently required during the energy-exposure treatment session;
use the lighting system to present to the breathing patient the third illumination to indicate that normal breathing will soon conclude and that controlled breathing will soon begin.

12. The apparatus of claim 11 wherein the first, second, and third illumination each comprise ambient illumination.

13. The apparatus of claim 11 further comprising:
a ceiling-mounted frame that supports the lighting system.

14. The apparatus of claim 13 wherein the ceiling-mounted frame disposes the lighting system at least 1.5 meters distant from the breathing patient.

15. The apparatus of claim 13 wherein the ceiling-mounted frame comprises an arc-shaped frame.

16. The apparatus of claim 13 wherein the apparatus further comprises at least one image-capture apparatus that is also supported by the ceiling-mounted frame.

17. The apparatus of claim 16 wherein the at least one image-capture apparatus comprises at least two image-capture apparatuses that are aimed and configured to capture images of the breathing patient.

18. The apparatus of claim 13 wherein the apparatus further comprises at least one audio transducer that is also supported by the ceiling-mounted frame.

19. The apparatus of claim 18 wherein the at least one audio transducer is operably coupled to the control circuit, and wherein the control circuit is configured to provide acoustic content to the breathing patient via the at least one audio transducer in accompaniment to at least one of presenting to the breathing patient the first illumination, the second illumination, and the third illumination.

20. A non-transitory computer-readable medium comprising instructions stored thereon for use during an energy-exposure treatment session of a target volume in a breathing patient, which target volume moves in response to the breathing, which instructions, when executed on a processor, perform the steps of:
presenting to the breathing patient a first illumination that corresponds to a first light modulation to indicate when normal breathing is allowed during the energy-exposure treatment session;
presenting to the breathing patient a second illumination that corresponds to a second light modulation that is different from the first light modulation to indicate that controlled breathing is required during the energy-exposure treatment session;
presenting to the breathing patient a third illumination that corresponds to a third light modulation that is different from both the first light modulation and the second light modulation to indicate that normal breathing will soon conclude and that controlled breathing will soon begin.
